SEQUENCE LISTING (4 sequences)

SEQ ID NO: 1 - DNA, 2838 bp, H. sapiens
  CDS (107)...(2369)
actcactatagggctcgagcggccgcccgggcaggtgaacttactacatcagaactcctgctgaggtataagaggatacgtctaataactcaattgctgtaaacctatgattgtttttctggtttttaaacatctcttcagcctcagattgattacaatgttctttctacttcatttcatcgttctgatcaatgtcaaagattttgcactgactcaaggtagcatgatcactccttcatgccaaaaaggatattttccctgtgggaatcttaccaagtgcttaccccgagcttttcactgtgatggcaaggatgactgtgggaacggggcggacgaagagaactgtggtgacactagtggatgggcgaccatatttggcacagtgcatggaaatgctaacagcgtggccttaacacaggagtgctttctaaaacagtatccacaatgctgtgactgcaaagaaactgaattggaatgtgtaaatggtgacttaaagtctgtgccgatgatttctaacaatgtgacattactgtctcttaagaaaaacaaaatccacagtcttccagataaagttttcatcaaatacacaaaacttaaaaagatatttcttcagcataattgcattagacacatatccaggaaagcattttttggattatgtaatctgcaaatattatatctcaaccacaactgcatcacaaccctcagacctggaatattcaaagacttacatcagctaacttggctaattctagatgacaatccaataaccagaatttcacagcgcttgtttacgggattaaattccttgtttttcctgtctatggttaataactacttagaagctcttcccaagcagatgtgtgcccaaatgcctcaactcaactgggtggatttggaaggcaatagaataaagtatctcacaaattctacgtttctgtcgtgcgattcgctcacagtgctgtttctgcctagaaatcaaattggttttgttccagagaagacattttcttcattaaaaaatttaggagaactggatctgtctagcaatacgataacggagctatcacctcacctttttaaagacttgaagcttctacaaaagctgaacctgtcatccaatcctcttatgtatcttcacaagaaccagtttgaaagtcttaaacaacttcagtctctagacctggaaaggatagagattccaaatataaacacacgaatgtttcaacccatgaagaatctttctcacatttatttcaaaaactttcgatactgctcctatgctccccatgtccgaatatgtatgcccttgacggacggcatttcttcatttgaggacctcttggctaacaatatcctcagaatatttgtctgggttatagctttcattacctgctttggaaatctttttgtcattggcatgagatctttcattaaagctgaaaatacaactcacgctatgtccatcaaaatcctttgttgtgctgattgcctgatgggtgtttacttgttctttgttggcattttcgatataaaataccgagggcagtatcagaagtatgccttgctgtggatggagagcgtgcagtgccgcctcatggggttcctggccatgctgtccaccgaagtctctgttctgctactgacctacttgactttggagaagttcctggtcattgtcttccccttcagtaacattcgacctggaaaacggcagacctcagtcatcctcatttgcatctggatggcgggatttttaatagctgtaattccattttggaataaggattattttggaaacttttatgggaaaaatggagtatgtttcccactttattatgaccaaacagaagatattggaagcaaagggtattctcttggaattttcctaggtgtgaacttgctggcttttctcatcattgtgttttcctatattactatgttctgttccattcaaaaaaccgccttgcagaccacagaagtaaggaattgttttggaagagaggtggctgttgcaaatcgtttcttttttatagtgttctctgatgccatctgctggattcctgtatttgtagttaaaatcctttccctcttccgggtggaaataccagacacaatgacttcctggatagtgatttttttccttccagttaacagtgctttgaatccaatcctctatactctcacaaccaacttttttaaggacaagttgaaacagctgctgcacaaacatcagaggaaatcaattttcaaaattaaaaaaaaaagtttatctacatccattgtgtggatagaggactcctcttccctgaaacttggggttttgaacaaaataacacttggagacagtataatgaaaccagtttcctagcaatcattttggatcactggactttcagtggactacctaaaacaggggacagcttttggaagatgacatctgcaatgcttttcatctttaccaacggcaagcctttctgcacagagagcacagcagaatggctcctgtcactgcattccaatggcagctgtactatctaccaaccatgctgaggacagcaccaaaggttcctctcctcaccccacatgcctgaaaagcacatgtgaattcgtgtatagtgggctgaggtgcagctgatctctagctaatcaacacaacccaccaacaaatgaccacaggttggcactgtgtggtctttcacatcgggttgcactgtccatgaaatagaaacactcacaacatctgattccagtgtggccataataacagaaatctaacaactctttccttgccttttcaatatcaaataaaaccatcagcatcctgctggattgata

SEQ ID NO: 2 - protein, 754 aa, H. sapiens
MIVFLVFKHLFSLRLITMFFLLHFIVLINVKDFALTQGSMITPSCQKGYFPCGNLTKCLPRAFHCDGKDDCGNGADEENCGDTSGWATIFGTVHGNANSVALTQECFLKQYPQCCDCKETELECVNGDLKSVPMISNNVTLLSLKKNKIHSLPDKVFIKYTKLKKIFLQHNCIRHISRKAFFGLCNLQILYLNHNCITTLRPGIFKDLHQLTWLILDDNPITRISQRLFTGLNSLFFLSMVNNYLEALPKQMCAQMPQLNWVDLEGNRIKYLTNSTFLSCDSLTVLFLPRNQIGFVPEKTFSSLKNLGELDLSSNTITELSPHLFKDLKLLQKLNLSSNPLMYLHKNQFESLKQLQSLDLERIEIPNINTRMFQPMKNLSHIYFKNFRYCSYAPHVRICMPLTDGISSFEDLLANNILRIFVWVIAFITCFGNLFVIGMRSFIKAENTTHAMSIKILCCADCLMGVYLFFVGIFDIKYRGQYQKYALLWMESVQCRLMGFLAMLSTEVSVLLLTYLTLEKFLVIVFPFSNIRPGKRQTSVILICIWMAGFLIAVIPFWNKDYFGNFYGKNGVCFPLYYDQTEDIGSKGYSLGIFLGVNLLAFLIIVFSYITMFCSIQKTALQTTEVRNCFGREVAVANRFFFIVFSDAICWIPVFVVKILSLFRVEIPDTMTSWIVIFFLPVNSALNPILYTLTTNFFKDKLKQLLHKHQRKSIFKIKKKSLSTSIVWIEDSSSLKLGVLNKITLGDSIMKPVS

SEQ ID NO: 3 - DNA, 3133 bp, H. sapiens
  CDS (1141)...(1330)
  CDS (2514)...(2629)
agactccctgtagtcccagctactcaggaggcagaggctgaaggatcgcttgagcccaggggaatgaggttacagtgagctatgatcctgccattacaggccaacccagagagatcctatgtcaaaaatgaataaataccctggtgtggtcgctctcgcctgtgatcccaggactttgggaggctcaggcgggaagatgacttgagtccaggagtttgagaccagcctgggcaacatggcaaaaccccatctctacaaaaaaaatacaaaaaattagccgggtgtggtggcgtgggcctgtagtcccagctactcaggaggctgaggtgggagaattgcttgagtcagggatagggaggtggcagtgagcccatatcgtgccactgcactccagccagggtaacagagcaagaactcatcacaaaaataaaaaataaaaataggtggctgggcgtggtggcttacacctgtaatcccagctacttgggaggctgaggtgggagaatcacttgaacccaggaggtggaggttgcagtgagccgagatcgcatcactgcactccagcctggaagacagagcaagactttgtctcaataataataataaaaataataatagtccaggcacagctcatgcctgtaatcccagcattttgggaggccgaggcagatggatcgcttgaggtcaggagttggagaccagcctggccaacatggcgaaccccagtctctactaaaaataaaaaataaaaaaaaaaaaattagccaggcatagtggtcgcacctgtaatcccagctactcaggaggctgaggcaggagaatcgcttgaacccgggtgtcagaagttgcagtgagccgagatcaagtcactgcactccaacctgggtgacagagtgaactccatctcaaaaaaaaagaagaaaaaaataaaaaagaaagactcgttgcccagtgctccctctgggagaagtacatccaaggccctggccctgggagaaaggctctggcactaaccccacccttgacctttttcctgggcggtcctgaagaatgttctgtcccttctccaaggcccccagctgggacggcccaggcgcctataaagggggtccccgcttggccccggcgccacccaccaccatggacccccgtctgcccgcctgggcgctggtgctgctgggccctgccctggtgttcgcgttgggccccgcgcccaccccagagatgcgtgagaagttgtgcggccaccacttcgtacgcgcgctggtgcgcgtgtgcgggggcccccgctggtccaccgaagccaggaggcctgcggccggaggcgaccgtgagtgggacgggcagggacagcgctctgggaagtccgaggtggggcaggtgcacgtaggcgcagatgcacacgtgcagggaggtgggcaggtttgcatgcatgagcccagggctcacctgccggctgcgtgcacagatcgtgggtccgcactggagtgtgtgtgaacgcgcgtgtgaaagtccccagagccgttagccaggagtggtggctcacgctgcaatcccagcactttgggaggctgaggcgggtggatcccaggtcgcactctagaggatccaggggaggtggtctgcaagggtgtgtcctcgtggggtgtgtgtgtgtctgtgggtccaccacacatgcgtgggtgaccaggaagtggacagacacatgtacacccctgcatgtctctgtaccacgaggagtctgtgggtgggtctctgacctgctctgggtgtctcacggacccttggacctgttgggaaactgacaccatctgtagctccaagacccccgacactccctgggcagggcatggggcatcctcaggtggcatatggggacccacgcagctgggatgtgagtgtggacatgagttgagtggatgagaggattaaccaggtgcctggcgtacagtaggtgcttaataagtgctcactttttttttttttttttttttttgagatggagtttcattcttgtcacctaggctggagtgcaatggcatgatcccagctccctgcaaccttccacctcctgggttcaagtgattctcccaccccagcctccccagtagctgggattacaggcgcccgcacacacccgctaattttttggtttttttttttttagtagagaaggggtttcactgtgttagccaggatggtctcaaactcctgacctggacgccacctgctcagactcgaggcatgcaaagtgctgggattacaggtgtaagccactgcacccggccagtgctcactttttatctgcatgcatgagtgtttggtgggttactcagtgggatgtggtggattcgtgcacatgcagcatggggtagcatgtcctctgtcgtttccgttccagctgagtttcactctgtccccaggtgagttgctacagtggctggagagacgacatctgctccatgggctggtggccgacagtaatctcacgctgggacctggcctgcagcccctgccccagacctctcaccatcaccgccaccaccgtgcagctgccaccaaccctgcacgctactgctgcctcagtggctgtacccaacaagacctgctgaccctctgtccctactgattcctccttgggtgcagcctcagagtggcctgaggcccagagggtctggtctggtgagctcctgaggccacacagcaccataaagtctcgcatctacaggcctttgattacctcctgggatgggtgctcactatctaccccagaccaatgccacctgcagcctgtggagtcaactgcagaataaatcacaccctagccctggcttggaggatccccgctttcacagatgctggacactgacagccaaatgtcctcactccagaggagccccagacgctccgctccctgcatgtgtaacacccctcttgctgtctcttagtaaataaacgacccaaagcagcttcctgcattttctctagaaaaagggacaagcctcagtcagtccctgtgtgggcttgtccccagcagccatggacccaccggcacttccaggagctttataactagagaggcattgcaacgcaagtggggaaactgaggcccagggagagggctgcttgcttg

SEQ ID NO: 4 - protein, 101 aa, H. sapiens
MDPRLPAWALVLLGPALVFALGPAPTPEMREKLCGHHFVRALVRVCGGPRWSTEARRPAAGGDLPQTSHHHRHHRAAATNPARYCCLSGCTQQDLLTLCPY

(19) United States
(12) Patent Application Publication
(10) Pub. No.: US 2005/0079180 A1
(43) Pub. Date: Apr. 14, 2005

(54) Manipulatin of oocyte maturation and male germ cell survival

(76) Inventors: Kazuhiro Kawamura, Stanford, CA (US); Aaron J.W. Hsueh, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/951,756

(22) Filed: Sep. 27, 2004

Related U.S. Application Data
(60) Provisional application No. 60/506,510, filed on Sep. 25, 2003.

(51) Int. Cl. A61K 48/00; A61K 39/395; A61K 31/56

(52) U.S. Cl. 424/146.1; 514/44; 514/171

(57) ABSTRACT

Compositions and methods are provided for modulating the survival and maturation of male and female mammalian germ cells. Compounds that interfere with the binding of INSL3 to its receptor, LGR8, are administered to an individual to prevent oocyte maturation, thereby acting as a contraceptive. INSL3 or INLS3 agonists are administered to an individual, or to cells in vitro, to enhance oocyte maturation, either for infertility treatment or for contraception. Compounds that interfere with the binding of INSL3 to its receptor are administered to an individual to enhance male germ cell apoptosis, thereby acting as a contraceptive. INSL3 or INLS3 agonists are administered to an individual, or to cells in vitro, to enhance sperm cell survival. LGR8 and INSL3 are useul separately or in combination in the screening and design of agonists and antagonists for use in the methods of the invention.

### BACKGROUND OF THE INVENTION

The growth and maturation of mammalian germ cells is intricately controlled by hormones; including gonadotropins secreted by the anterior pituitary; and local paracrine factors. The majority of the oocytes within the adult human ovary are maintained in prolonged stage of first meiotic prophase; enveloped by surrounding follicular somatic cells. Periodically, a group of primordial follicles enters a stage of follicular growth. During this time, the oocyte undergoes a large increase in volume, and the number of follicular granulosa cells increase. The maturing oocyte synthesizes paracrine factors that allow the follicle cells to proliferate, and the follicle cells secrete growth and differentiation factors (for example TGF-β2, VEGF, leptin, and FGF2) that enhance angiogenesis and allow the oocyte to grow. After progressing to a certain stage, oocytes and their follicles die, unless they are exposed to gonadotropic hormones that prevent somatic cell apoptosis and induce meiotic maturation in the oocyte.

In early stages of the cycle, maturing follicles respond to follicle stimulating hormone (FSH) with further growth and cellular proliferation. FSH also induces the formation of luteinizing hormone (LH) receptors on the granulosa cells. During the cycle, the pituitary also secrets LH. LH binds specific receptors in ovarian theca and mature granulosa cells. In response to LH, the oocyte meiotic block is broken. The nuclear membranes of competent oocytes break down, and the chromosomes assemble to undergo the first meiotic division. In the male, LH secreted by the pituitary binds to specific receptors in the testis Leydig cells. LH also plays an important role in spermatogenesis. Up to 75% of the germ cells in testis are deleted during spermatogenesis, and LH is a survival factor for these cells.

In addition to pituitary hormones, other factors are involved in these processes. Because LH acts exclusively on somatic cells in the gonads, local paracrine factors are likely involved in the regulation of oocyte meiosis arrest and optimal spermatogenesis. INSL3, also know as Leydig insulin-like hormone or relaxin-like factor, was originally named based on its exclusive expression in Leydig cells of fetal and adult testes. However, INSL3 is also expressed in thecal and luteal cells of the ovary. Male INSL3 null mice exhibit bilateral cryptorchidism whereas female INSL3 null mice show impaired fertility. Recent studies indicated that testis INSL3 acts as an endocrine factor to activate a G protein-coupled receptor, LGR8, the gubernaculum with increases in cAMP production (Kumagai et al. (2002) J Biol Chem 277:31283-31286).

The importance of oocyte and male germ cell maturation make the development of agents to modulate these processes of great clinical interest. The present invention addresses these issues.

### SUMMARY OF THE INVENTION

Compositions and methods are provided for modulating the survival and maturation of male and female mammalian germ cells. The present invention demonstrates that INSL3 is a paracrine hormone in both female and male gonads and interacts with its receptor LGR8 expressed in oocytes and male germ cells to activate an inhibitory guanine nucleotide-binding protein. In vitro and in vivo treatment with INSL3 initiates meiotic progression of arrested oocytes. Treatment with INSL3 suppresses male germ cell apoptosis.

In one embodiment of the invention, compounds that interfere with the binding of INSL3 to its receptor are administered to an individual to prevent oocyte maturation, thereby acting as a contraceptive. In another embodiment of the invention, INSL3 or INLS3 agonists are administered to an individual, or to cells in vitro, to enhance oocyte maturation. In a contraceptive method, tt is also possible to induce premature maturation of the oocyte in a few large follicles with the potential to become the preovulatory follicle during the early follicular phase of the cycle. After the mid-cycle LH surge that induces the rupture of the preovulatory follicle, an oocyte that is released has premature maturation and has reduced capacity for fertilization and further development into embryos. In such methods, INSL3 may be administered for a short period of time in the cycle, e.g. one week or less, down to a single dose in the cycle.

In another embodiment of the invention, compounds that interfere with the binding of INSL3 to its receptor are administered to an individual to enhance male germ cell apoptosis, thereby acting as a contraceptive. In another embodiment of the invention, INSL3 or INLS3 agonists are administered to an individual, or to cells in vitro, to enhance sperm cell survival.

LGR8 and INSL3 are useul separately or in combination in the screening and design of agonists and antagonists for use in the methods of the invention.

### BRIEF DESCRIPTION OF THE FIGURES

FIGS. 1A-1G. Expression of LGR8 in the ovary and INSL3 stimulation of oocyte maturation. A) Northern blot analysis of ovarian LGR8 transcripts in gonadotropin-treated rats. B) RT-PCR amplification of LGR8 transcripts in oocyte but not granulosa cells. GAPDH serves as a positive control. C) In situ hybridization localization of LGR8 in the oocyte. D) INSL3 augmentation of germinal vesicle breakdown of cultured cumulus-enclosed oocytes. E) Time-dependency of INSL3 augmentation of oocyte maturation. F) INSL3 stimulation of oocyte maturation in cultured follicles. G) Inhibitory effects of pertussis toxin on INSL3 and LH induction of oocyte maturation.

FIGS. 2A-2D. Expression of LGR8 in male germ cells and INSL3 activation of the inhibitory G protein in seminiferous tubules. A) Northern blot analyses of LGR8 expression in testes of developing rats. B) In situ hybridization localization of LGR8 transcripts in the testis. The antisense LGR8 or INSL3 probe was used for the analysis of testes from six-week-old rats. Hybridization signals for LGR8 and INSL3 were present in seminiferous tubules and interstitial cells, respectively. C) Direct binding of biotinylated INSL3 to different testicular compartments. T: total binding; N: nonspecific binding; S: specific binding. LGR8: 293T cells expressing recombinant LGR8. Control: 293T cells not transfected with the LGR8 plasmid. D) Pertussis toxin (PT) pretreatment blocked the inhibitory effect of INSL3 on cAMP production. Seminiferous tubular cells were treated with different hormones or forskolin (FSK) for 12 h before cAMP determination. Some cells were also pretreated with PT before hormonal treatment (filled circles) to suppress Gi activity. B: basal.

FIGS. 3A-3D. Stimulation of ovarian INSL3 expression by hCG during the preovulatory period, and INSL3 induction of oocyte maturation but not follicle rupture in PMSG-primed rats. A) Increases in INSL3 mRNA levels following hCG treatment. Quantitative RT-PCR was performed using ovarian samples before and after treatment of immature rats with PMSG (15 IU, two days), followed by administration of 10 IU hCG at different intervals. B) In situ hybridization analysis of INSL3 expression in preovulatory follicles at 1.5 h after hCG treatment of PMSG-primed rats. C) In vivo treatment with INSL3-induced oocyte maturation and luteinized unruptured follicles. PMSG-primed rats were treated with INSL3, and oviducts were examined one day later for ovulated oocytes. (D) Morphology of oocytes induced by INSL3 to undergo maturation (arrows).

FIGS. 4A-D. Treatment with hCG increased INLS3 expression whereas treatment with INSL3 suppressed testis germ cell apoptosis induced by a GnRH antagonist. A) Changes in testis and other organ weights following treatment with the GnRH antagonist with or without INSL3 or hCG. Immature rats at 28 days of age were treated daily with a GnRH antagonist (AN; Antagon, 25 μg/day) with or without hCG (75 IU/day) or synthetic rat INSL3 (2 μg, twice daily) for five days before organ removal. B) Northern blot analyses of testis INSL3 mRNA levels in rats treated with the GnRH antagonist with or without hCG. C) Analysis of apoptotic DNA fragmentation in testes using 3′-end DNA labeling with the terminal transferase, followed by gel fractionation and autoradiography. D) In situ staining of apoptotic DNA fragmentation in seminiferous tubules using the TUNNEL method. Positive signals are evident in some primary spermatocytes in control animals (panel d, light arrows) whereas treatment with the GnRH antagonist (AN) led to additional signals in meiotic germ cells (panel e, heavy arrows). Testis from animals with both INSL3 and the GnRH antagonist showed staining similar to the control group.

FIG. 5. Gonadotropin stimulation of oocyte maturation and male germ cell survival involves an intra-gonadal paracrine system mediated by INSL3 and its receptor, LGR8. Pituitary LH stimulates theca or Leydig cells to produce INSL3, which, in turn, activates LGR8 receptors and the inhibitory G protein in oocytes to induce meiotic maturation or, in male germ cells, to suppress apoptosis.

### DESCRIPTION OF THE SPECIFIC EMBODIMENTS

Compositions and methods are provided for modulating the survival and maturation of mammalian germ cells. By modulating the effects of the paracrine hormone, INSL3, oocyte maturation and survival of male germ cells can be manipulated. Compounds that interfere with the binding of INSL3 to its receptor are administered to an individual to prevent oocyte maturation or sperm cell apoptosis, thereby acting as a contraceptive. INSL3 or INLS3 agonists are administered to an individual, or to cells in vitro, to enhance oocyte maturation or sperm survival for infertile patients. Premature induction of oocyte maturation also serves as the basis for a once-a-month, non-steroidal contraceptive approach. The INSL3 receptor, LGR8; and INSL3 find use in the screening and design of agonists and antagonists for use in the methods of the invention.

The methods of the invention find use in a wide variety of animal species, particularly including mammalian species. Animal models, particularly small mammals, e.g. murine, lagomorpha, etc. are of interest for experimental investigations. Other animal species may benefit from improvements in in vitro fertilization, e.g. horses, cattle, rare zoo animals such as panda bears, large cats, etc. Humans are of particular interest for both enhancing oocyte activation, and for methods of contraception.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

Oocyte and Oocyte maturation. The meiotic division of mammalian oocytes begins with one primary germ cell (oocyte), which gives rise to only one mature ovum (egg). In normal mammalian development, oocytes become developmentally arrested in the ovaries at the germinal vesicle stage in prophase of the first meiotic division (G2/M transition). In response to the mid-cycle LH surge, oocytes of preovulatory follicles resume meiosis. The first meiotic division is completed with the extrusion of a diploid set of chromosomes into the first polar body, another diploid set of chromosomes remaining within the cytoplasm of the oocyte. The cells then proceed to the second meiotic division, where the oocyte becomes arrested at metaphase (“Met II”). Met II oocytes are mature, and can be ovulated and fertilized.

Meiotic arrest of the oocyte is most likely maintained by follicular purines that increase cAMP levels in the oocyte. A decrease in intra-oocyte cAMP levels is required for meiotic resumption in oocytes. Meiotic arrest is released following injection of an antibody against the stimulatory G protein. The effect of LH on oocytes is shown herein to be mediated through the paracrine factor INSL3, which is produced by follicular theca cells in response to LH. INSL3 has a direct stimulatory effect on oocyte maturation, causing the oocytes to resume meiosis. The evidence provided herein demonstrates that follicle rupture (somatic cells) and oocyte maturation (germ cells) can be separated, providing a basis for contraception trhough blocking the interaction between INSL3 and its receptor, LGR8 expressed exclusively in the oocyte.

Male Germ Cell. Male germ cells include the various cells in the sperm lineage, including A and B type spermatogonia; primary and secondary spermatocytes, which complete the second division of meiosis; and haploid spermatids and sperm. During the process of spermiogenesis, spermatids mature into sperm comprising an acrocome and flagella.

In the testis, most male germ cells undergo apoptosis. INSL3, like LH, is a survival factor for male germ cells. The present findings demonstrate that INSL3 binds to seminiferous tubules by interacting with the LGR8 receptor expressed in meiotic germ cells to activate the Gi protein. There is a coordinate increase of expression of INSL3 in Leydig cells of the testis. Treatment with INSL3 suppresses male germ cell apoptosis.

INSL3. INSL3 beling to the insulin-like hormone superfamily, which group also includes comprises relaxin, INSL4, INSL5, INSL6, insulin, and insulin-like growth factors I and II. The members of this family are characterized by a signal peptide, a B-chain, a connecting C-peptide, and an A-chain. Previously it has been reported that INSL3 was expressed exclusively in prenatal and postnatal Leydig cells. See, for example, Adham et al. (1993) J. Biol. Chem. 268:26668-26672; and Burkhardt et al. (1994) Genomics 20: 13-19.

The INSL3 gene comprises 2 exons and 1 intron, having an organization similar to that of insulin and relaxin. The transcription start site is localized 14 bp upstream of the translation start site. The human genome contains a single copy of the INSL3 gene. For convenience, the gene, coding sequences, and amino acid sequence are provided in SEQ ID NO:3 and SEQ ID NO:4. From the cDNA sequence, it has been deduced that INSL3 protein is synthesized as a 131-amino-acid (aa) preproprotein and that it contains a 24-aa signal peptide. Comparison of the pro INSL3 protein with members of the insulin-like hormone superfamily predicts that the biologically active hormone, after proteolytic processing of the C peptide, consists of a 31-aa long B chain and a 26-aa long A chain, and that it has a molecular weight of 6.25 kDa.

LGR8. LGR8 is a member of the G-protein coupled, seven trans-membrane family of proteins, specifically the subfamily of G-protein coupled seven trans-membrane proteins that are characterized by the presence of extra-cellular leucine rich repeat regions. LGR8 is described in co-pending patent application U.S. Ser. No. 10/222,668, herein incorporated by reference. The protein has both a G-protein coupled seven trans-membrane region and a leucine rich repeat extra-cellular domain. Both of these receptors mediate the production of cAMP in response to binding of relaxin, which production is inhibited by the addition of anti-relaxin antibodies.

For convenience, the sequence of LGR8 is provided as SEQ ID NO:1, and encodes a polypeptide of 754 amino acids (SEQ ID NO:2). LGR8 is mainly expressed in the brain, kidney, muscle, testis, thyroid, uterus, bone marrow and peripheral blood cells. In addition to INSL3, LGR8 is a relaxin receptor. INSL3 has been reported to be expressed exclusively in prenatal and postnatal Leydig cells in males. In the ovary, INSL3 is expressed in theca cells and luteal cells.

### Contraceptive Methods

The specificity of INSL3 expression, and its role in maturation of oocytes, makes it an attractive candidate for contraceptive design. Agents that interfere with the biological activity of INSL3 can prevent oocyte maturation, potentially without affecting follicle rupture and normal menstrual cycle. INLS3 inhibitors of interest for these purposes include agents that bind to INSL3 and prevent it from acting in its biological role; agents that bind to the INSL3 receptor, LGR8, which agents may act as competitive inhibitors of INSL3; and agents that otherwise act as an INSL3 inhibitor or antagonist. Competitive binding antagonists, for example, a polypeptide that mimics INSL3 binding may be used to inhibit activity. Other inhibitors are identified by screening for biological activity, e.g. in a binding assay based on the receptor ligand interaction; binding to the INSL3 ligand; inhibition of the LGR8 receptor; and the like.

INSL3 inhibitors may be administered locally or systemically to female individuals for contraceptive purposes; in a dose effective to prevent the maturation of oocytes. The compounds may be administered on a daily, weekly, or semi-weekly schedule for all or a portion of the menstrual cycle. The compounds may also be formulated for sustained release, e.g. in a monthly implant; semi-yearly implant; and the like.

INSL3 inhibitors may also be administered to male individuals for contraceptive purposes, in a dose effective to cause apoptosis of male germ cells sufficient to create infertility. The compounds may be administered on a daily, weekly, or semi-weekly schedule. The compounds may also be formulated for sustained release, e.g. in a monthly implant; semi-yearly implant; and the like.

INSL3 may also be administered for contraceptive purposes by timing the administration so as to cause premature oocyte maturation in the preovulatory follicles, leading to contraception.

### Methods of Enhancing Fertility

Agonists of INLS3, including INSL3 itself and mimetics, analogs and variants thereof, agents that bind to LGR8; and in in vitro culture, e.g. in combination with in vitro fertilization techniques. The dose will be effective to permit oocytes to pass to maturation, i.e. to proceed to the second meiotic division, and arrest at metaphase (“Met II”), which oocytes can be ovulated and fertilized. For in vitro purposes the INSL3 agonist may replace follicular cells, which would otherwise produce the factor.

INSL3 agonists may also find use in enhacing the survival of sperm, e.g. for enhancement of germ cell survival. INSL3 agonists may be administered locally or systemically to male individuals; in a dose effective to allow sperm survival sufficient for fertility. The compounds may be administered on a daily, weekly, or semi-weekly schedule. For such purposes it may not be required to administer the agent for extended periods of time.

### Modulation of LGR8 Activity

In one embodiment of the invention, genetic agents are used to modulate expression of INSL3 or LGR8 for contraception or enhancement of fertility. The genetic sequences, gene fragments, or antisense sequences are useful in gene therapy. Inhibition can be achieved in a number of ways. Antisense or siRNA sequences may be administered to inhibit expression. Upregulating activity is also of interest, for example through the introduction of mutations that have a gain of function mutation, through increasing expression levels, through administering agents that bind to and activate LGR8, etc.

Expression vectors may be used to introduce the INSL3 or LGR8 gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The INSL3 or LGR8 sequence may be introduced into tissues or host cells by any number of routes, including viral infection, microinjection, or fusion of vesicles. Jet injection may also be used for intramuscular administration, as described by Furth et al. (1992) Anal Biochem 205:365-368. The DNA may be coated onto gold microparticles, and delivered intradermally by a particle bombardment device, or “gene gun” as described in the literature (see, for example, Tang et al. (1992) Nature 356:152-154), where gold microprojectiles are coated with the LGR8 or DNA, then bombarded into skin cells.

Antisense molecules can be used to down-regulate expression of INSL3 or LGR8 in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of such molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

In addition to antisense, small interfering RNA (siRNA) duplexes can be used to inhibit expression of jeb genes. siRNA are double stranded RNA molecules of at least about 18 nucleotides, and may be up to the length of the complete mRNA. Preferred siRNA for use in mammalian cells are from about 18 to 30 nucleotides, preferably from about 21 to 22 nucleotides in length. For example, see Elbashir et al. (2001) Nature 411:494-498.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars, heterocyclic bases, morpholino derivatives, and the like.

Agents that block INSL3 or LGR8 activity provide a point of intervention in an important signaling pathway, and are useful as contraceptives. Numerous agents are useful in reducing INSL3 or LGR8 activity, including agents that directly modulate INSL3 or LGR8 expression as described above, e.g. expression vectors, anti-sense specific for INSL3 or LGR8; and agents that act on the INSL3 or LGR8 protein, e.g. specific antibodies and analogs thereof, small organic molecules that block activity, etc.

### Compound Screening

Compound screening may be performed using an in vitro model, a genetically altered cell or animal, or purified protein corresponding to INSL3 (SEQ ID NO:4) or LGR8 (SEQ ID NO:2). One can identify ligands that bind to, inhibit, modulate or mimic the action of the encoded polypeptide. Screening for INSL3 agonsists is of particular interest.

Polypeptides useful in screening include those encoded by SEQ ID NO:1 and SEQ ID NO:3, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or a region associated with a consensus sequence). Variants also include fragments of the polypeptides discussed herein, particularly biologically active fragments and/or fragments corresponding to functional domains. Fragments of interest will typically be at least about 10 aa to at least about 15 aa in length, usually at least about 50 aa in length, and can be as long as 100 aa in length or longer, but will usually not exceed about 300 aa in length, where the fragment will have a contiguous stretch of amino acids that is identical to a polypeptide encoded by SEQ ID NO:1 and SEQ ID NO:3, or homologs thereof.

Transgenic animals or cells derived therefrom are also used in compound screening. Transgenic animals may be made through homologous recombination, where the normal locus corresponding to INSL3 or LGR8 is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different domains in signal transduction, etc. Of interest is the use of INSL3 or LGR8 to construct transgenic animal models for oocyte maturation and development; and for male germ cell maturation and survival, where expression of the gene is specifically reduced or absent. Specific constructs of interest include antisense sequences that block expression of INSL3 and expression of dominant negative mutations. A detectable marker, such as lac Z may be introduced into the locus of interest, where up-regulation of expression will result in an easily detected change in phenotype. One may also provide for expression of the gene or variants thereof in cells or tissues where it is not normally expressed or at abnormal times of development. By providing expression of the target protein in cells in which it is not normally produced, one can induce changes in cell behavior.

Compound screening identifies agents that modulate function of INSL3 or LGR8, particularly with respect to their effect on germ cell survival or oocyte maturation. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains, binding sites, and the like.

The term “agent” as used herein describes any molecule, e.g. protein or pharmaceutical, with the capability of altering, inhibiting, or mimicking the physiological function of INSL3 or LGR8. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Candidate agents of interest also include peptides and derivatives thereof, e.g. high affinity peptides or peptidomimetic ligands for LGR8, mimetic of INSL3 that bind to its receptor but do not activate signaling, agents that block INSL3 binding to LGR8, and the like.

Generally, peptide agents encompassed by the methods provided herein range in size from about 3 amino acids to about 100 amino acids, with peptides ranging from about 3 to about 25 being typical and with from about 3 to about 12 being more typical. Peptide agents can be synthesized by standard chemical methods known in the art (see, e.g., Hunkapiller et al., Nature 310:105-11, 1984; Stewart and Young, Solid Phase Peptide Synthesis, 2^{nd} Ed., Pierce Chemical Co., Rockford, Ill., (1984)), such as, for example, an automated peptide synthesizer. In addition, such peptides can be produced by translation from a vector having a nucleic acid sequence encoding the peptide using methods known in the art (see, e.g., Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999); which are incorporated by reference herein).

Peptide libraries can be constructed from natural or synthetic amino acids. For example, a population of synthetic peptides representing all possible amino acid sequences of length N (where N is a positive integer), or a subset of all possible sequences, can comprise the peptide library. Such peptides can be synthesized by standard chemical methods known in the art (see, e.g., Hunkapiller et al., Nature 310:105-11, 1984; Stewart and Young, Solid Phase Peptide Synthesis, 2^{nd} Ed., Pierce Chemical Co., Rockford, Ill., (1984)), such as, for example, an automated peptide synthesizer. Nonclassical amino acids or chemical amino acid analogs can be used in substitution of or in addition into the classical amino acids. Non-classical amino acids include but are not limited to the D-isomers of the common amino acids, α-amino isobutyric acid, 4-aminobutyric acid, 2-amino butyric acid, γ-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methy1 amino acids, C α-methyl amino acids, N α-methyl amino acids, and amino acid analogs in general. Furthermore, the amino acid can be D (dextrorotary) or L (levorotary).

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used. The components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to INSL3 or LGR8, as at least some of the compounds so identified are likely INSL3 or LGR8 inhibitors. The binding assays usually involve contacting INSL3 or LGR8 with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. The INSL3 or LGR8 protein utilized in such assays can be naturally expressed, cloned or synthesized.

Certain screening methods involve screening for a compound that modulates the expression of INSL3 or LGR8. Such methods generally involve conducting cell-based assays in which test compounds are contacted with one or more cells expressing INSL3 or LGR8 and then detecting and an increase in expression (either transcript or translation product). Some assays are performed with germ cells that express endogenous LGR8, or woth follicular cells that express INSL3.

INSL3 or LGR8 gene expression can be detected in a number of different ways. The expression level of INSL3 or LGR8 in a cell can be determined by probing the mRNA expressed in a cell with a probe that specifically hybridizes with a probe or transcript. Probing can be conducted by lysing the cells and conducting Northern blots or without lysing the cells using in situ-hybridization techniques. Alternatively, INSL3 or LGR8 protein can be detected using immunological methods in which a cell lysate is probe with antibodies that specifically bind to INSL3 or LGR8.

Other cell-based assays are reporter assays conducted with cells that do not express INSL3 or LGR8. Certain of these assays are conducted with a heterologous nucleic acid construct that includes an INSL3 or LGR8 promoter that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, CAT (chloramphenicol acetyl transferase; Alton and Vapnek (1979) Nature 282:864-869), luciferase, β-galactosidase and alkaline phosphatase (Toh, et al. (1980) Eur. J. Biochem. 182:231-238; and Hall et al. (1983) J. Mol. Appl. Gen. 2:101).

In these assays, cells harboring the reporter construct are contacted with a test compound. A test compound that either activates the promoter by binding to it or triggers a cascade that produces a molecule that activates the promoter causes expression of the detectable reporter. Certain other reporter assays are conducted with cells that harbor a heterologous construct that includes a transcriptional control element that activates expression INSL3 or LGR8 and a reporter operably linked thereto. Here, too, an agent that binds to the transcriptional control element to activate expression of the reporter or that triggers the formation of an agent that binds to the transcriptional control element to activate reporter expression, can be identified by the generation of signal associated with reporter expression.

The level of expression or activity can be compared to a baseline value. The baseline value can be a value for a control sample or a statistical value that is representative of INSL3 or LGR8 expression levels for a control population. Expression levels can also be determined for cells that do not express INSL3 or LGR8 as a negative control. Such cells generally are otherwise substantially genetically the same as the test cells.

Various controls can be conducted to ensure that an observed activity is authentic including running parallel reactions with cells that lack the reporter construct or by not contacting a cell harboring the reporter construct with test compound. Compounds can also be further validated as described below.

Active test agents identified by the screening methods described herein that modulate INSL3 or LGR8 activity can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

Once analogs have been prepared, they can be screened using the methods disclosed herein to identify those analogs that exhibit an increased ability to modulate INSL3 or LGR8 activity. Such compounds can then be subjected to further analysis to identify those compounds that appear to have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

### Antibodies Specific for INSL3 or LGR8 Polypeptides

Antibodies specific for INSL3 or LGR8 or epitopic fragments thereof may be used in the methods of the invention. As used herein, the term “antibodies” includes antibodies of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions, is a term well understood in the art, and indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for an INSL3 or LGR8 polypeptide.

Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

### Pharmaceutical Compositions

Compounds identified by the screening methods described above and analogs thereof can serve as the active ingredient in pharmaceutical compositions formulated for the treatment of various neurological disorders, including stroke. The compositions can also include various other agents to enhance delivery and efficacy. The compositions can also include various agents to enhance delivery and stability of the active ingredients.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which consist of the packaged active ingredient with a suppository base. Suitable suppository bases include natural or synthetic triglycerides or paraffin hydrocarbons. In addition, it is also possible to use gelatin rectal capsules, which consist of a combination of the packaged active ingredient with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

In pharmaceutical dosage forms, the compounds may be administered in the form of their pharmaceutically acceptable salts, or they may also be used alone or in appropriate association, as well as in combination with other pharmaceutically active compounds. The following methods and excipients are merely exemplary and are in no way limiting.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant is placed in proximity to the targeted site, so that the local concentration of active agent is increased relative to the rest of the body.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 μg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

### Induction of Oocyte Maturation and Male Germ Cell Survival Mediated by the Intra-Gonadal INSL3/LGR8 Paracrine System

In mammals, meiotic progression in the mature oocyte and survival of the meiotic male germ cells are indirectly dependent on exposure to luteinizing hormone (LH). LH does not directly interact with germ cells, but as shown herein, induces the activity of INSL3. The hormone INSL3 interacts with its receptor LGR8 expressed in oocytes and male germ cells to activate an inhibitory guanine nucleotide-binding protein. In vitro and in vivo treatment of with INSL3 initiates meiotic progression of arrested oocytes. Treatment with INSL3 suppresses male germ cell apoptosis.

In addition, induction of ovulation with gonadotropin leads to an increase in ovarian INSL3 expression, and co-treatment with pertussis toxin blocks the gonadotropin induction of oocyte maturation without affecting follicle rupture. Thus, INSL3 represents a meiosis-inducing factor in the ovary and mediates the survival action of gonadotropins on male germ cells.

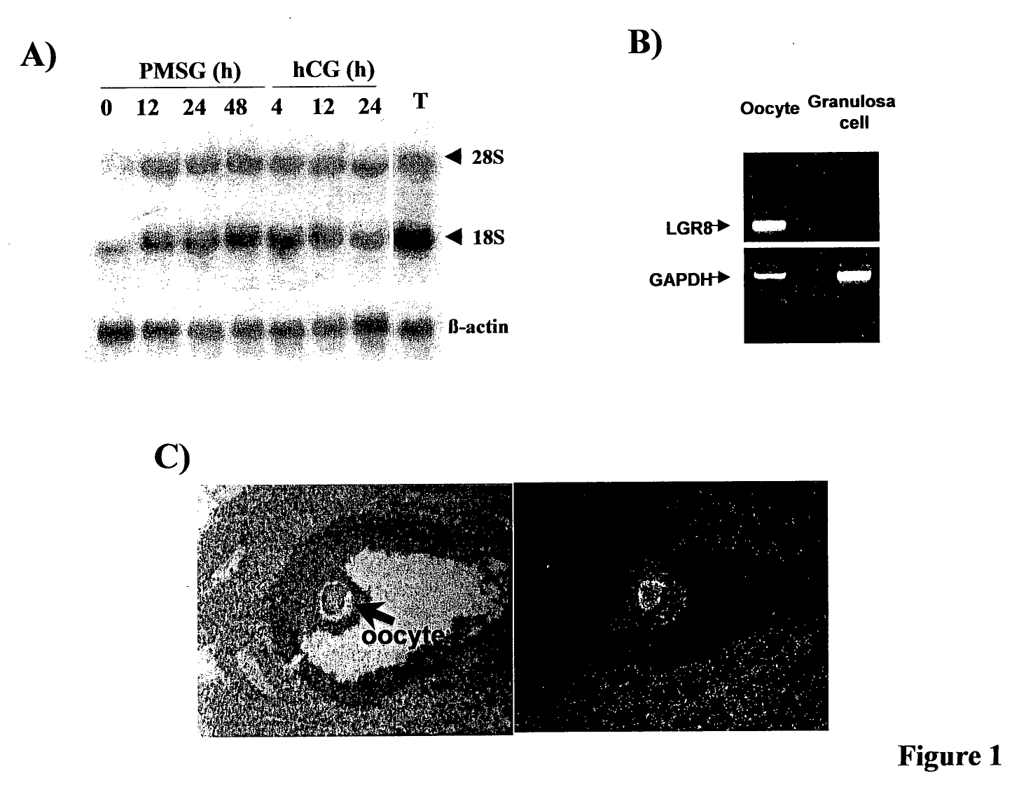

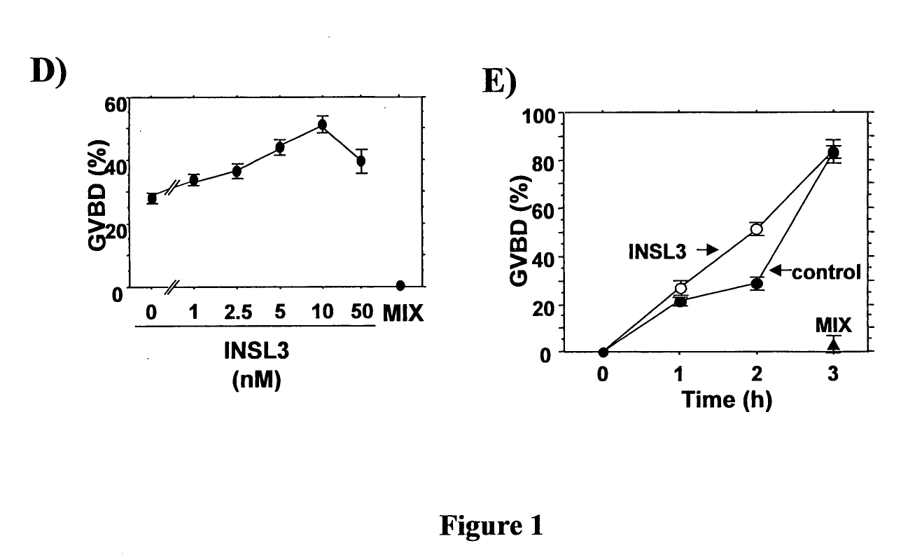

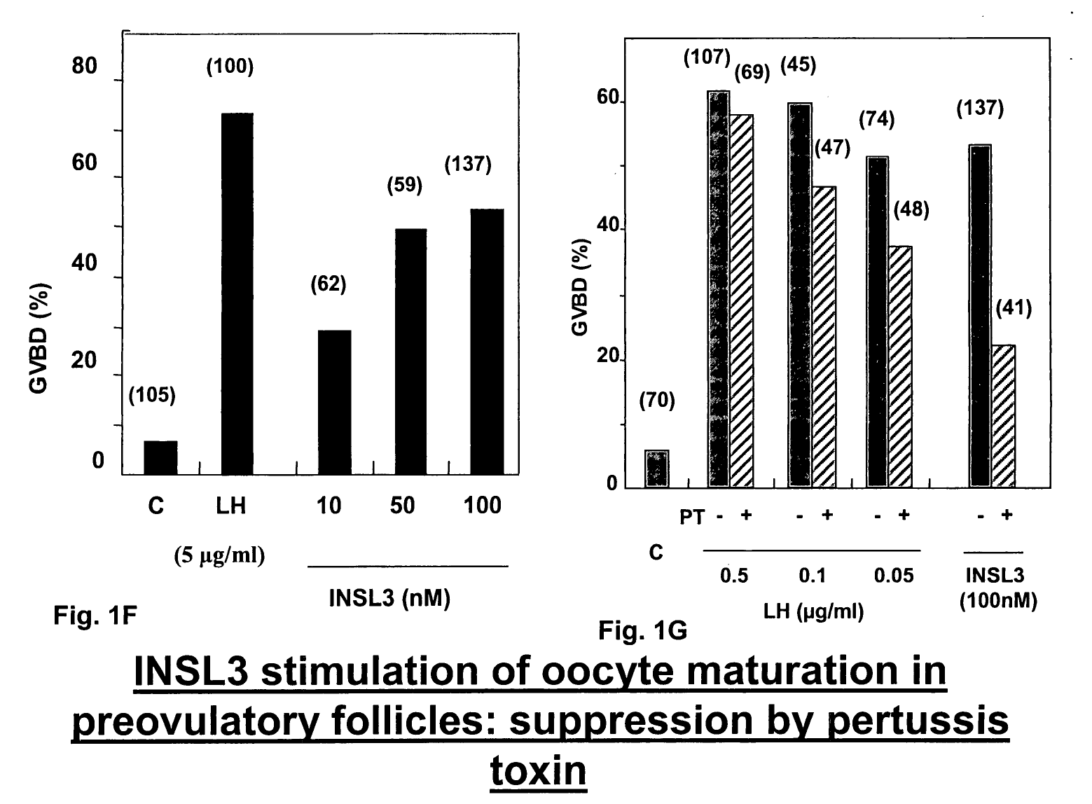

Northern blot analyses have indicated the LGR8 expression in ovaries of gonadotropin-treated rats (FIG. 1A). RT-PCR analyses and in situ hybridization studies further demonstrated the exclusive oocyte localization of LGR8 in the ovary (FIGS. 1B and 1C). We further tested the ability of INSL3 to modulate oocyte maturation. Although spontaneous meiotic resumption, evidenced by the breakdown of the nuclear membrane (germinal vesicle), could be observed in cultured cumulus-enclosed oocytes obtained from preovulatory follicles, treatment with INSL3 further augmented oocyte maturation in a dose-dependent manner (FIG. 1D). In contrast, inclusion of a phosphodiesterase inhibitor (MIX) completely blocked oocyte maturation.

Time-course studies indicated that the augmentation effects of INSL3 were only evident at 2 h after culture (FIG. 1E). When tested using intact preovulatory follicles, treatment with INSL3, like LH, induced oocyte maturation in a dose-dependent manner (FIG. 1F). Because oocyte maturation is associated with decreases in intra-oocytic cAMP levels, we hypothesized that the LH treatment stimulates endogenous INSL3, which in turn, induces oocyte maturation by activating the inhibitory guanine nucleotide-binding protein (Gi). As shown in FIG. 1G, treatment with pertussis toxin, a Gi inhibitor, partially blocked the LH induction of oocyte maturation at lower (50 and 100 ng/ml), but not higher, doses of LH (FIG. 1G). Treatment with pertussis toxin also partially blocked the INSL3 induction of oocyte maturation.

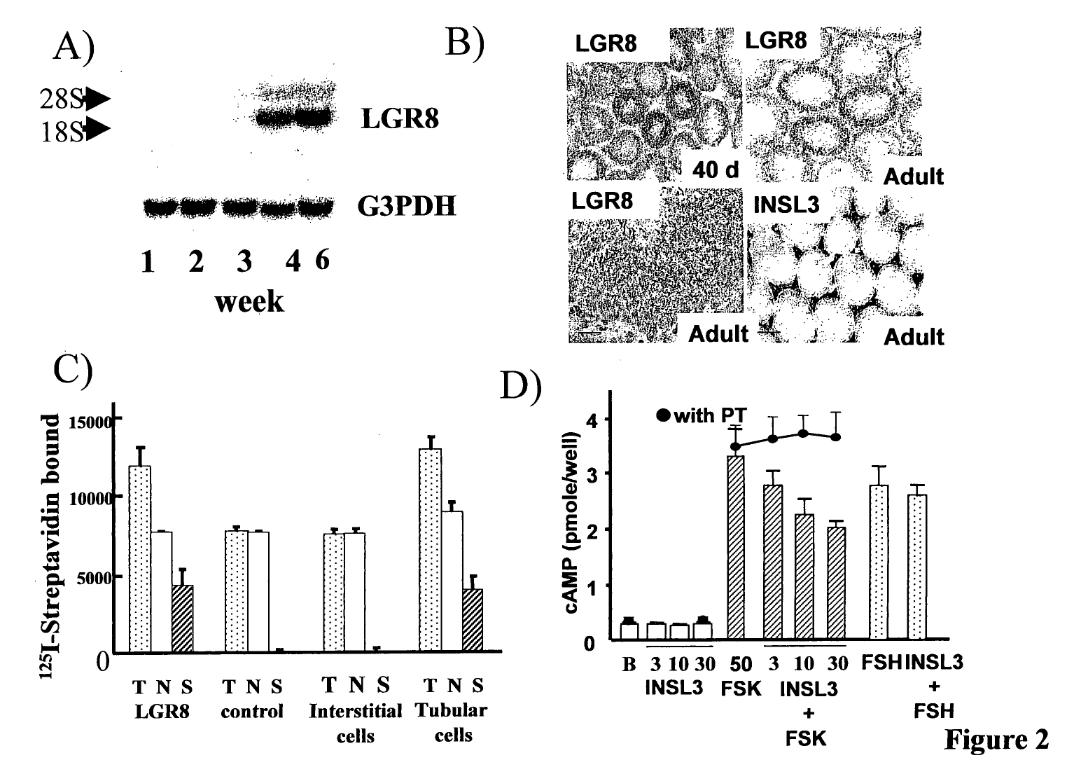

A Northern blot analysis of testes from developing rats was performed for INSL3 expression. A predominant 2.5 kb transcript was found in the testes of rats at four and six weeks of age but was undetectable in younger animals (FIG. 2A). In situ hybridization analyses further demonstrated that LGR8 is expressed in seminiferous tubules and not in the interstitial cells of rats at 40 and 60 days of age (FIG. 2B). Under higher magnification, hybridization signals could be found in meiotic germ cells including spermatocytes, but not in spermatogonia. In contrast, INSL3 expression was restricted to interstitial cells. Using biotinylated INSL3 as a tracer, INSL3 binding was found in seminiferous tubular cells but not interstitial cells (FIG. 2C). Seminiferous tubular cells were treated with INSL3 to activate LGR8. Although basal cAMP production was not affected, INSL3 treatment prevented cAMP increases induced by forskolin in a dose-dependent manner (FIG. 2D). Furthermore, the suppressive effect of INSL3 was blocked by pretreatment with pertussis toxin. To rule out the direct action of INSL3 on Sertoli cells, tubular cells were treated with FSH with or without INSL3. Although FSH stimulated cAMP production by Sertoli cells in the tubular cells, co-treatment with INSL3 did not alter cAMP levels (FIG. 2D).

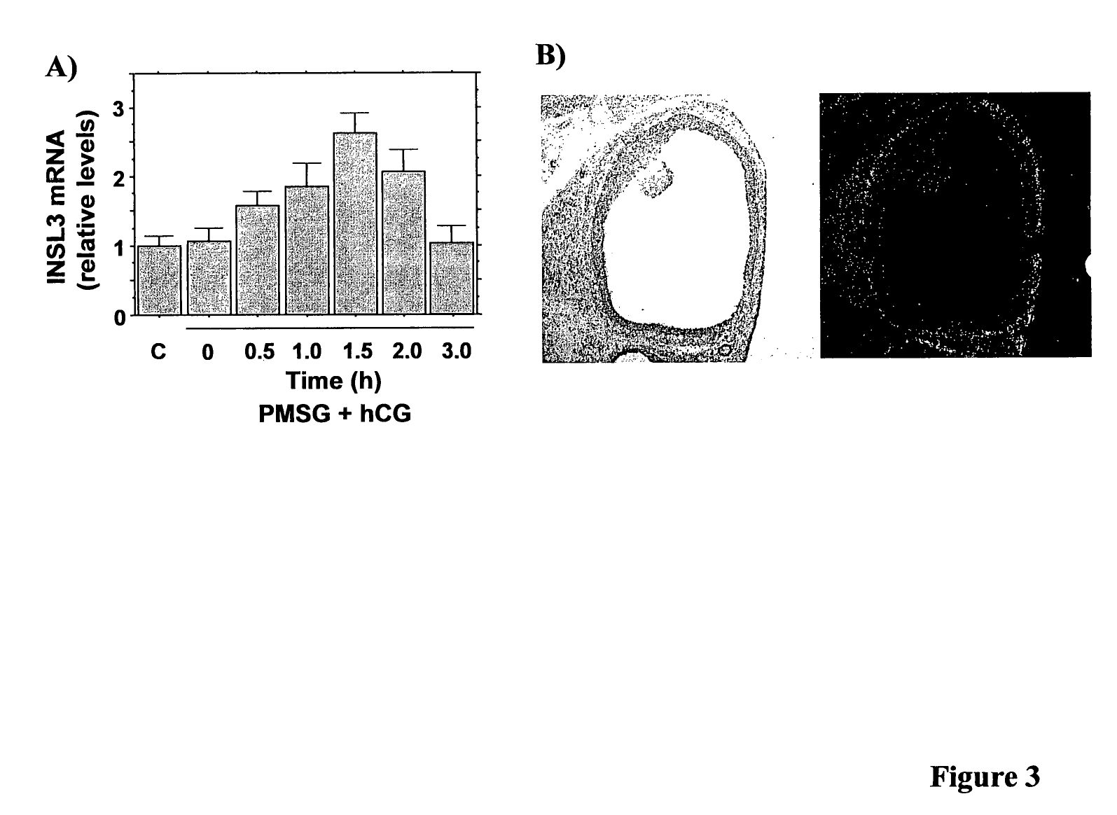

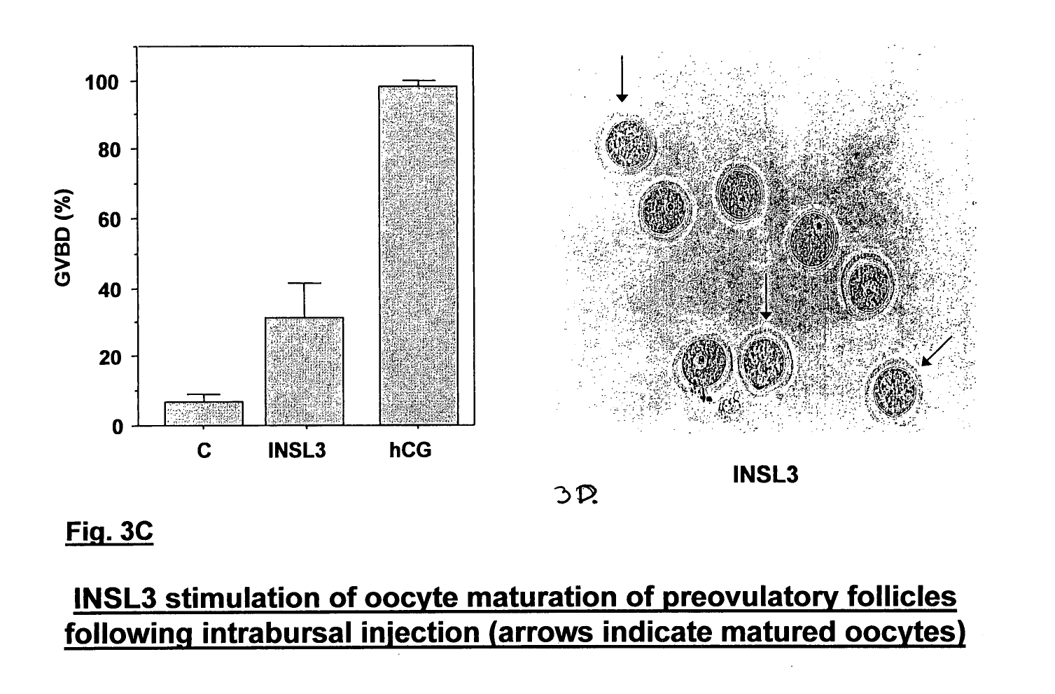

In the ovary, RT-PCR analyses indicated that INSL3 transcript levels increased at 1.5 h after treatment with an ovulatory dose of hCG, an agonist of LH, in immature rats primed with PMSG (FIG. 3A). In situ hybridization analyses confirmed the expression of INSL3 in theca cells surrounding the preovulatory follicles (FIG. 3B). In addition, intrabursal treatment of PMSG-primed rats with INSL3, like hCG, induced oocyte maturation but not follicle rupture as revealed by germinal vesicle breakdown found in preovulatory follicles at 8 h after intrabursal injection of INSL3 (FIG. 3C).

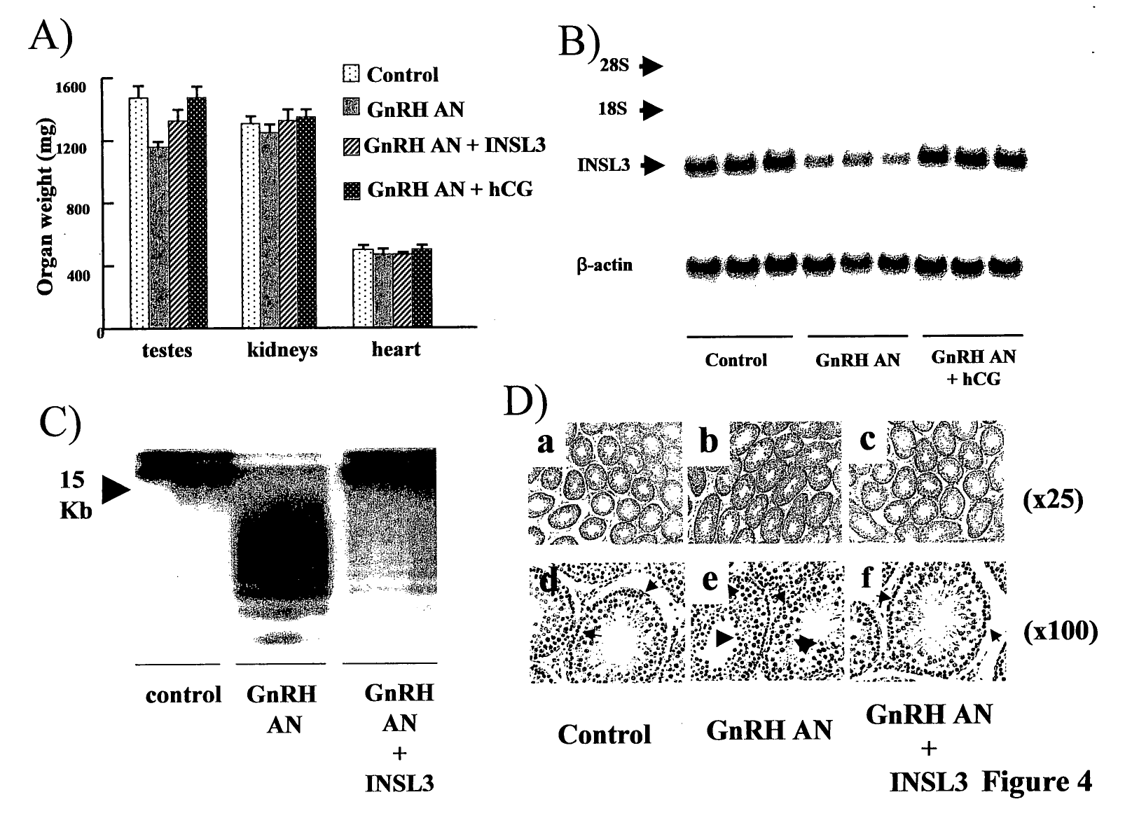

The paracrine role of INSL3 in the testis was tested using a gonadotropin deprivation model (Tapanainen et al. (1993) Mol Endocrinol 7:643-650). Immature rats were treated for five days with a GnRH antagonist with or without hCG or INSL3. GnRH antagonist treatment decreased testis weight; however, this inhibitory effect was partially blocked by co-treatment with INSL3 or hCG (FIG. 4A). In contrast, no weight change in kidney or heart was detected. Northern blot analyses indicated that treatment with the GnRH antagonist decreased INSL3 message levels whereas treatment with hCG increased INSL3 expression (FIG. 4B). As shown in FIG. 4C, treatment with the GnRH antagonist increased apoptotic DNA fragmentation but co-treatment with INSL3 blocked apoptosis based on the analysis of internucleosomal DNA fragmentation. In situ staining of DNA fragments further indicated that, in control animals, spermatogenesis had advanced to elongation phase spermatids and a number of tubules were delineated in their periphery by apoptotic round germ cells identified as primary spermatocytes (FIG. 4D, panel d). After treatment with GnRH antagonist, the majority of tubules showed labeled cells corresponding to meiotic pachytene spermatocytes and postmeiotic spermatids (panel e). In contrast, co-treatment with INSL3 blocked the apoptosis-inducing action of the GnRH antagonist (panel f).

Mammalian oocytes exhibit prolonged arrest in the meiotic prophase (G2/M transition). In response to the mid-cycle LH surge, oocytes of preovulatory follicles resume meiosis, proceeding to the metaphase of the second meiotic division. The dissolution of the nuclear membrane is followed by the extrusion of the first polar body. Meiotic arrest of the oocyte is most likely maintained by follicular purines that increase cAMP levels in the oocyte, however changes in hypoxanthine levels could not be found before the oocyte maturation induced by gonadotropin. Although LH stimulates cAMP production in follicular somatic cells, a decrease in intra-oocyte cAMP levels is required for meiotic resumption in oocytes. Indeed, meiotic arrest is released following injection of an antibody against the stimulatory G protein.

The present results of a transient stimulation of INSL3 expression in theca cells by LH prior to oocyte maturation, and the direct stimulatory effects of INSL3 on oocyte maturation, suggest a paracrine role of the INSL3-LGR8 system in the resumption of meiosis. Although meiosis-activating sterols (MAS) induce oocyte maturation in culture, the exact role of MAS in mediating LH-stimulated oocyte maturation remains controversial. FSH treatment also stimulates a paracrine factor from cumulus cells to promote oocyte maturation, but the nature of this factor is unknown. In addition to confirming INSL3 expression in thecal cells, we demonstrate that an ovulatory dose of hCG increases INSL3 expression, consistent with a paracrine role of INSL3.

The ovulatory process consists of oocyte maturation, follicle rupture, and luteinization. Earlier studies indicated that cycling rats treated with inhibitors for the oocyte-specific phosphodiesterase 3 enzyme maintained normal cycling and follicle rupture but ovulated oocytes were immature and not fertilizable. The present study further confirms the possibility of separating follicle rupture and oocyte maturation, thus providing the basis to develop contraceptives using LGR8 blockers.

In the testis, most male germ cells undergo apoptosis, perhaps as a mechanism to delete superfluous or defective germ cells. INSL3, like LH, is a survival factor for male germ cells. Observed stimulation of INSL3 transcripts in the testis by hCG is consistent with earlier studies using hypogonadal mice. Our findings demonstrate that INSL3 binds to seminiferous tubules by interacting with the LGR8 receptor expressed in meiotic germ cells to activate the Gi protein. In situ hybridization studies indicate the expression of LGR8 in meiotic germ cells of the testis, consistent with observed increases in LGR8 transcripts between three and four weeks of age when the germ cells progress through the first round of meiosis. Observed developmental increases in LGR8 expression in rat testes coincides with similar changes of the INSL3 transcript in mouse Leydig cells, suggesting a coordinate expression of the INSL3/LGR8 paracrine genes during male germ cell progression.

In contrast to the stimulatory effects of INSL3 on cAMP production by gubernacular cells and by 293T cells expressing recombinant LGR8, LGR8 expressed in germ cells is coupled to the Gi protein. It appears that G protein coupling of LGR8 is dependent on the specific cell types in which they are expressed. Indeed, many of the GPCRs can signal through different classes of G proteins.

In both female and male germ cells, a complete GPCR/G protein/adenyl cyclase/phosphodiesterase system is present. Decreases in cAMP levels play an important role in the meiotic arrest of female germ cells. Oocyte maturation is regulated by antibodies to Gs and phosphodiesterase 3 inhibitors. Rat oocyte also expresses adenyl cyclase AC3 and the inhibitory G proteins. Although no functional GPCRs have been found in the oocyte, adenosine A3 receptor and several odorant receptors are present in male germ cells and implicated in germ cell maturation and chemotaxis. The male germ cells also express an olfactory type of the Gα subunit protein, adenyl cyclase III, and phosphodiesterases. In male germ cells, a low level of cAMP maintained by INSL3 may be important for meiotic progression and increases in cAMP following INLS3 deprivation may lead to apoptosis.

Female INSL3 null mice exhibited impaired fertility associated with increases in follicular atresia and premature luteolysis. In contrast, female LGR8 null mice are fertile, suggesting the existence of additional pathways in the regulation of oocyte maturation. For males, deletion of both INSL3 and LGR8 genes led to bilateral cryptorchidism due to defective gubernaculum development during embryogenesis. Impaired spermatogenesis found in adults was attributed to the secondary effects of undescended testes, and surgical correction of cryptorchid testes in the INSL3 null mice partially corrected male infertility. Because paralogous genes for INSL3 are expressed in the testis, optimal spermatogenesis could be regulated by redundant signaling systems.

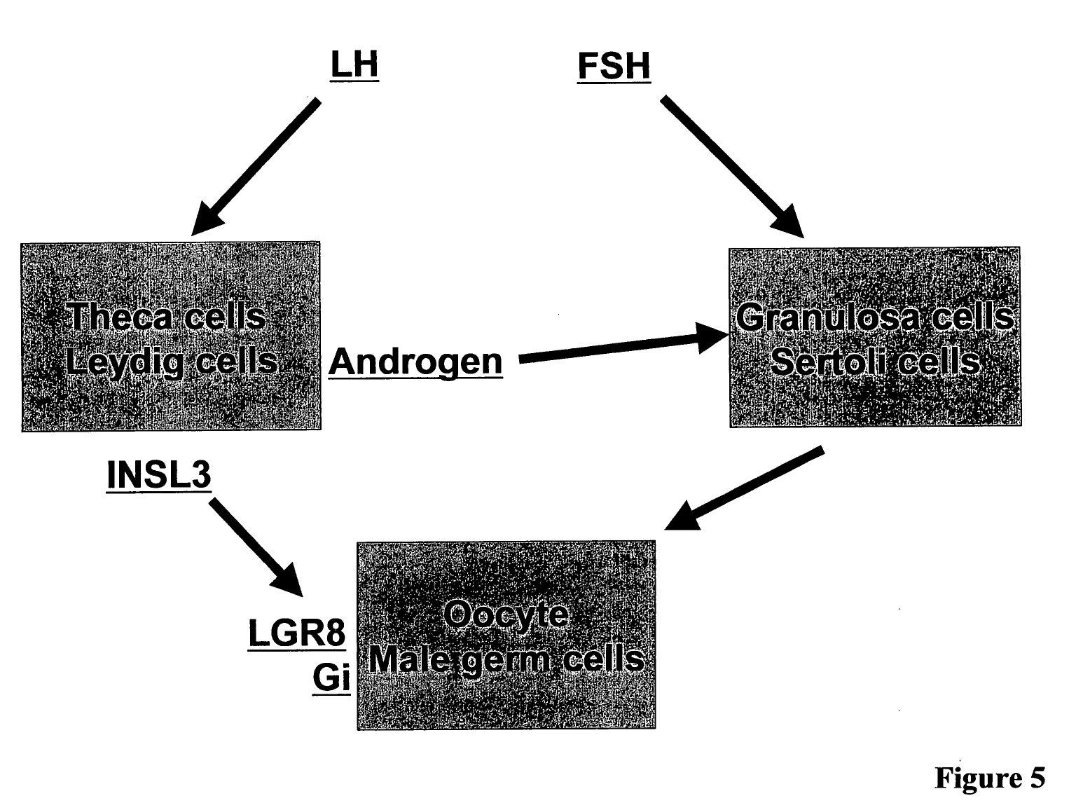

A unifying picture of the gonadotropin regulation of germ cell function is emerging (FIG. 5). LH, in addition to promoting androgen production by ovarian thecal cells and testis Leydig cells, stimulates INSL3 biosynthesis. INSL3, in turn, activates the Gi-coupled LGR8 to initiate oocyte maturation and suppress male germ cell apoptosis. Although INSL3 is secreted into general circulation and peaks before parturition, the present data highlights its paracrine role. Because LGR8 is mainly expressed in gonadal tissues during adulthood, future screening of agonistic or antagonistic ligands for this INSL3 receptor may facilitate the discovery of new contraceptive strategies or novel treatments for infertility.

Methods:

Rat and ovine INSL3 were chemically synthesized and characterized as described Smith et al. (2001) J Pept Sci 7:495-501. Biotinylated ovine INSL3 contains an additional biotin molecule on the N-terminus of the A chain. The National Hormone and Pituitary Program (NIDDK, National Institutes of Health, Bethesda, Md.) supplied porcine relaxin. GnRH antagonist ganirelix acetate (Antagon™) and human FSH were from Organon (West Orange, N.J.) whereas hCG was from A.P.L, Ayerst Laboratories Inc. (Philadelphia, Pa.). ¹²⁵I-streptavidin and streptavidin conjugated to horseradish peroxidase (HRP) were purchased from Amersham Biosciences, Inc (Piscataway, N.J.), whereas foskolin, collagenase, and trypsin were from Sigma Chemical Co. (St. Louis, Mo.). Sprague-Dawley rats were obtained from Simonsen Laboratories (Gilroy, Calif.). Animal care was consistent with institutional and NIH guidelines.

Total RNA from rat testes were extracted using the RNeasy purification kits (Qiagen Inc. Chatsworth, Calif.) before Northern blotting. The rat ortholog for LGR8 was identified in the GenBank (accession number AC098990) and this sequence was used in reverse transcription-PCR to yield a LGR8 probe of 230 bp for Northern blotting and in situ analyses. For in situ hybridization studies, testes form 40 days old Sprague-Dawley rat testes were fixed at 4° C. for 6 h in 4% paraformaldehyde in PBS, followed by immersion in 0.5 M sucrose in PBS overnight. Cryostat sections (7 μm thick) were mounted on microscope slides coated with poly-L-lysine (Sigma Chemical Co., St. Louis, Mo.) and stored at −80C. until analyzed. The hybridization procedure was essentially the same as described by Leo et al. (1999) Endocrinology 140:5469-5477. In brief, sections were pretreated serially with 0.2 M HCl, 2×SSC, pronase E (0.125 mg/ml), 4% paraformaldehyde, acetic anhydride in triethanolamine and dehydrated in ascending grades of ethanol. The antisense and sense probes were labeled with [³⁵S]UTP (1,000 Ci/mmol, Amersham, Piscataway, N.J.) and sections were hybridized overnight at 45° C. in 50% formamide, 0.3 M NaCl, 10 mM Tris-HCl, 5 mM EDTA, 1× Denhardt's solution, 10% dextran sulfate, 1 μg/ml carrier transfer RNA, and 10 mM dithiothreitol. Following ribonuclease A (20 μg/ml) treatment at 37° C. for 30 min, posthybridization washing was performed to a final stringency of 0.1×SSC. Slides were dipped into NTB-2 emulsion (Eastman Kodak Co., Rochester, N.Y.) and exposed at 4° C. for 1 week before development. The slides were stained with hematoxylin and eosin and mounted with DPX Mountant (Electron Microscopy Sciences, Ft. Washington, Pa.). The slides were photographed using a Zeiss 35 mm camera and microscope (MC80DC; Carl Zeiss, Oberkochen, Germany) with bright- and darkfield illumination.

Seminiferous tubular cells were isolated by the method of Nagao (1989) In Vitro Cell Dev Biol 25:1088-1098, with modifications. Briefly, testes were decapsulated and incubated for 15 minutes in PBS containing 0.25% collagenase with occasional shaking. After collecting interstitial cells released from the preparation, seminiferous tubules were washed, and incubated with 0.25% trypsin to disperse tubular cells. The resulting cell suspension was filtered through 100 μm nylon mesh to remove cell aggregates and tissue debris, after which the tubular cells were collected by centrifugation. For INSL3 binding studies, cells were suspended in D-PBS containing 1% BSA and incubated with biotinylated ovine INSL3 (10 nM/tube) with or without increasing doses of the rat INSL3 at 4° C. for 24 h. After incubation, cells were centrifuged and washed twice with 1% BSA/PBS before incubation with ¹²⁵I-streptavidin (400,000 cpm/tube) for 1 h at 4° C. After washing the cells three times, radioactivity in the pellets was determined.

To express recombinant LGR8, human 293T cells derived from embryonic kidney fibroblasts were maintained in Dulbecco's modified Eagle's medium/Ham's F-12 (DMEM/F12) supplemented with 10% FBS, 100 μg/ml penicillin, 100 μg/ml streptomycin, and 2 mM L-glutamine. Cells (2×10⁶/culture) were transfected using the calcium phosphate precipitation method and used for hormonal stimulation as described by Kumagai et al. (2002) J Biol Chem 277:31283-31286. Total cAMP in each well was measured in triplicate with primary cultured cells by a specific radioimmunoassay as previously described by Davoren and Hsuch (1985) Biol Reprod 33:37-52. All experiments were repeated at least four times.

Male Sprague-Dawley rats at 28 days of age were treated subcutaneously with GnRH antagonist (250 μg/kg/day) to suppress pituitary gonadotropin secretion. Some animals were treated with the GnRH antagonist together with subcutaneous injections of hCG (75 IU/day), FSH (30 IU/12 h) or synthetic rat INSL3 (1 μg/12 h). Animals were killed 5 days after initial treatment and organ weight determined. Testes were stored at −70° C. before RNA or DNA extraction or fixed in Bouin's solution for in situ labeling of DNA ends.

DNA from whole testis was isolated and quantitated with spectrophotometry at 260 nm. Aliquots of DNA (500 ng) from each sample were labeled at 3′-ends with 32P-dideoxy-ATP (3,000 Ci/mmol: Amersham. Arlington Heights, Ill.) using terminal transferase (25 U/sample; Boehringey-Mannheim, Indianapolis, Ind.) as described. Labeled samples were fractionated through 2% agarose gels. After electrophoresis, gels were dried for 2 h in a slab-gel dryer without heat and exposed to Kodak X-OMAT AR films (Eastman Kodak Co., Rochester, N.Y.) at −70 C. After autoradiography, portions of each lane corresponding to DNA less than 10 kilobases (kb) were isolated and counted in a y-spectrometer for estimating the degree of intenucleosomal DNA fragmentation. For in situ DNA 3′-end labeling, fixed testicular tissues were embedded in paraffin, and cut in 3 μm sections. To detect apoptotic DNA fragmentation, the in situ cell death detection kit (Roche Diagnostics, Mannheim, Germany) was used according to manufacturer's instruction and sections counterstained with hematoxylin.

Cumulus-enclosed oocytes (CEO) were collected and cultured as described (Vaknin et al. PMID: 11133687). Briefly, female rats at 26 d of age were injected with 15 IU pregnant mare serum gonadotropin (PMSG). After 48-50 h, ovaries were dissected in Leivovitz's L-15 medium (Invitrogen Corp., Carlsbad, Calif.) supplemented with 5% fetal bovine serum and 100 μg/ml of penicillin and streptomycin. The CEOs were collected by puncturing the largest ovarian follicles in the L15 medium containing 0.25 mM of MIX within 20 min, washed twice in L15 medium without MIX and transfer to test medium. The CEO were cultured with or without different doses of rat INSL3 in L15 medium without both FBS and IBMX. For controls, the CEOs were cultured in L15 medium with IBMX. At the end of culture, the occurrence of germinal vesicle breakdown (GVBD) in oocyte was examined after removing cumulus cells surrounding CEO by a small-bore pipette under Hoffman modulation contrast microscopy (Nikon Inc., Tokyo, Japan). For preovulatory follicles, follicles were excised from the ovary after PMSG treatment for 48 h. The follicles, 20-30 per vial, were cultured with or without different doses of rat INSL3 or LH in L15 medium without FBS. The vials were flushed at the start of the culture with O₂/N₂ (1/1) and cultured at 37C with gently shaking. After culture, individual CEO was dissected from each follicle to examine the occurrence of GVBD. For cAMP determination, 20 to 30 denuded oocytes were cultured with or without rat INSL3 (10 nM) in L15 medium containing 10 μM of cilostamide with or without 50 μM of forskolin. After treatment, oocytes were collected in 2 μl of medium, frozen and thawed to break the cells before acetylation of samples with triethylamine and acetic anhydride (3:1). Intra-oocyte cyclic AMP levels were determined by radioimmunoassay as previously described (Davoren, Hsuch, 1985).

To study the effect of INSL3 on oocyte maturation in vivo, intrabursal injection was performed (Vaknin et al. PMID: 11133687). Immature PMSG-treated rats were lightly anesthetized by ether, and one of the ovaries was exteriorized through a small lumbosacral incision. Rat INSL3, dissolved in 100 μl of PBS, was injected through a 30-gauge needle threaded into the ovarian bursa via the adjoining fat pad. After injection, the ovary was replaced into the abdominal cavity, and skin was clipped. Eight hours later, CEOs were collected by puncturing the largest ovarian follicles to examine the occurrence of oocyte GVBD.

All publications and patent applications cited in this specification are herein incorporated by reference as if each individual publication or patent application were specifically and individually indicated to be incorporated by reference. The publications discussed herein are provided solely for their disclosure prior to the filing date of the present application. Nothing herein is to be construed as an admission that the invention is not entitled to antedate such a disclosure by virtue of prior invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of contraception, the method comprising:
administering an inhibitor of INSL3 or LGR8 to an individual in a dose effective to prevent germ cell maturation or survival.

2. The method according to claim 1, wherein said individual is a female, and said inhibitor is administered at a dose effective to prevent oocyte maturation.

3. The method according to claim 2, wherein said inhibitor blocks the binding of INSL3 to LGR8.

4. The method according to claim 3, wherein said inhibitor is an antibody.

5. The method according to claim 3, wherein said inhibitor is a small organic molecule.

6. The method according to claim 2, wherein said inhibitor decreases expression of INSL3.

7. The method according to claim 6, wherein said inhibitor is an siRNA or an anti-sense oligonucleotide.

8. A method of enhancing fertility, the method comprising:
contacting a mammalian germ cell with an agent that enhances INSL3 signaling, in a dose effective to enhance germ cell maturation or survival.

9. The method according to claim 8, wherein said agent is INSL3 or a derivative or mimetic thereof.

10. The method according to claim 8, wherein said mammalian germ cell is an oocyte, and said agent is provided in an in vitro culture.

11. The method according to claim 8, wherein mammalian germ cell is in the sperm cell lineage and said agent is administered to a male individual.

12. The method according to claim 8, wherein said mammalian germ cell is an oocyte, and said agent is administered to a female individual.

13. A method of screening for contraceptives, the method comprising:
combining a candidate biologically active agent with any one of:
(a) an LGR8 or INSL3 polypeptide;
(b) a cell comprising a nucleic acid encoding an LGR8 or INSL3 polypeptide; or
(c) a non-human transgenic animal model comprising an exogenous and stably transmitted LGR8 or INSL3 gene; and
determining the effect of said agent on signaling by INSL3.

14. The method according to claim 13, further comprising:
determining the effect of said agent on germ cell maturation or survival.

15. The method according to claim 14, wherein said germ cell is an oocyte.

16. The method according to claim 14, wherein said germ cell is a cell in the sperm cell lineage.

17. A method of screening for an agent that enhances germ cell maturation or survival, the method comprising:
combining a candidate biologically active agent with any one of:
(a) an LGR8 or INSL3 polypeptide;
(b) a cell comprising a nucleic acid encoding an LGR8 or INSL3 polypeptide; or
(c) a non-human transgenic animal model comprising an exogenous and stably transmitted LGR8 or INSL3 gene; and
determining the effect of said agent on signaling by INSL3.

18. The method according to claim 17, further comprising:
determining the effect of said agent on germ cell maturation or survival.

19. The method according to claim 18, wherein said germ cell is an oocyte.

20. The method according to claim 18, wherein said germ cell is a cell in the sperm cell lineage.

21. A method of contraception, the method comprising:
administering an agonist of INSL3 to an individual in a dose effective to cause premature oocyte maturation and blocking the capacity of the oocyte for maturation.

22. The method of contraception according to claim 21, wherein said agonist of INSL3 is INSL3 or a derivative or mimetic thereof.

23. A method of designing biologically active agents that modulate INSL3 function, the method comprising:
determining the binding site between LGR8 and INSL3;
designing a pharmacomimetic molecule that mimics the binding site interaction.
